(19) United States
(12) Patent Application Publication
(10) Pub. No.: US 2016/0030544 A1
(43) Pub. Date: Feb. 4, 2016

(54) IMMUNOGENIC COMPOSITION TO NEISSERIA

(71) Applicant: ISIS INNOVATION LIMITED, Oxford Oxfordshire (GB)

(72) Inventors: Christoph Marcel Tang, Oxford (GB); Susan Mary Lea, Oxford (GB); Ilse Jongerius, Oxford (GB); Hayley Lavender, Oxford (GB)

(73) Assignee: Isis Innovation Limited, Oxford (GB)

(21) Appl. No.: 14/776,229

(22) PCT Filed: Mar. 12, 2014
(86) PCT No.: PCT/GB2014/050726

§ 371 Date: Sep. 14, 2015

Related U.S. Application Data
(60) Provisional application No. 61/784,933, filed on Mar. 14, 2013.

(51) Int. Cl. A61K 39/095; C07K 14/22

(52) U.S. Cl. CPC A61K 39/095; C07K 14/22

(57) ABSTRACT

The present invention provides an immunogenic composition capable of eliciting an immune response when administered to a human or non-human animal, wherein the composition comprises an isolated protein with one or more of the following properties: i) about 70, 75, 80, 85, 90, 91, 92, 93, 94, 95, 96, 97, 98, 99 or more percent sequence identity to the protein of SEQ ID No: 1 or a fragment, derivative or analog thereof; ii) is a modified factor H binding protein, wherein the factor H binding protein has been modified at least at the position equivalent to position 318 as defined in FIG. 6 (SEQ ID No: 2); iii) does not bind to factor H; and iv) the immune response elicited is cross reactive with two or more of variant 1 factor H binding protein, variant 2 factor H binding protein and variant 3 factor H binding protein from N. meningitidis; and uses thereof.

The present invention relates to immunogenic compositions for use in eliciting immune responses to pathogenic organisms, and in particular, to immunogenic compositions capable of eliciting protective immune responses.

Neisseria meningitidis (meningococcus—N. meningitidis) is an encapsulated gram-negative diplococcus bacterium that inhabits the nasopharynx of up to 40% of healthy humans. The complex host-pathogen relationship is usually of a commensal nature. Occasionally, however, meningococcal carriage can lead to invasive disease. It is also a leading cause of sepsis and meningitis in very young children and adolescents with a case fatality rate of around 20%. The nonspecific early symptoms and the rapid development of disease mean that there is an urgent need for vaccine development to prevent meningococcal sepsis. N. meningitidis is classified in thirteen serogroups based on the composition of the polysaccharide capsule, but only six serogroups are responsible for disease. At the moment there are successful vaccines based on the polysaccharide capsule against five of these serogroups (A, C, W135, X and Y). A vaccine for serogroup B (MenB) cannot be based on its polysaccharide capsule which is composed of α2-8 linked polysialic acid because it is structurally identical to a modification of cell adhesion molecules that are present in the foetal brain. Therefore, the serogroup B capsule is poorly immunogenic and could induce autoimmunity if used as vaccine. Vaccines based on outer membrane vesicles have proven to be effective against MenB but only in combating epidemic disease by a single clone. Current research shows that the most efficient way to produce a broad protective vaccine against all N. meningitidis strains (including MenB) will be the use of protein based vaccines.

N. meningitidis subverts the immune response of a host organism by mimicking the host. N. meningitidis uses protein, in the form of the factor H binding protein (fHbp), instead of charged-carbohydrate chemistry to recruit the host complement regulator, factor H. In healthy individuals, the activation of complement is precisely controlled through membrane-bound and soluble plasma-regulatory proteins including factor H (fH). Factor H is a 155 kDa protein composed of twenty domains (termed complement control protein repeats, or CCPs). N. meningitidis, like several pathogens, have adapted to avoid complement-mediated killing by sequestering Factor H to their surface.

An aim of this invention is to provide one or more compositions which can be used to elicit a protective immune response against N. meningitidis, and in particular a protective immune response against N. meningitidis serogroup B. In particular, the present invention may provide one or more compositions capable of eliciting an immune response directed to the fHbp of N. meningitidis, and thus prevent, or reduce, the binding of factor H to the fHbp protein, and thereby to prevent or reduce N. meningitidis subverting the hosts immune response, or to result in direct killing of the bacterium in the presence of complement or phagocytic cells.

According to a first aspect, the present invention provides an immunogenic composition capable of eliciting an immune response when administered to a human or non-human animal, wherein the composition comprises an isolated protein with one or more of the following properties:
i) about 70, 75, 80, 85, 90, 91, 92, 93, 94, 95, 96, 97, 98, 99 or more percent sequence identity to the protein of SEQ ID No: 1;
ii) is a modified factor H binding protein, wherein the factor H binding protein has been modified at least at the position equivalent to position 318 as defined in FIG. 6 (SEQ ID No: 2);
iii) does not bind to factor H; and
iv) the immune response elicited is cross reactive with two or more of variant 1 factor H binding protein, variant 2 factor H binding protein and variant 3 factor H binding protein from N. meningitidis.

The protein of SEQ ID No: 1 is a protein derived from Neisseria gonorrhoeae. It is an intracellular protein, so would not naturally be considered as a vaccine target. The protein is referred to herein as Ghfp (Gonnococcal homologue of the factor H binding protein) or SEQ ID No: 1.

Preferably, an immunogenic composition according to the invention comprises an isolated protein with one or more of the following properties:
i) about 70, 75, 80, 85, 90, 91, 92, 93, 94, 95, 96, 97, 98, 99 or more percent sequence identity to the protein of SEQ ID No: 1;
iii) does not bind to factor H; and
iv) the immune response elicited is cross protective against strains expressing variant 1 factor H binding protein, variant 2 factor H binding protein and variant 3 factor H binding protein from N. meningitidis.

In a preferred embodiment the isolated protein has about 95% or more sequence identity with the protein of SEQ ID No: 1.

Preferably the isolated protein in the composition does not bind to factor H.

Preferably the immune response elicited by the composition is cross reactive to variant 1, 2 and 3 factor H binding proteins from N. meningitidis.

In an embodiment the isolated protein in the composition has about 70, 75, 80, 85, 90, 91, 92, 93, 94, 95, 96, 97, 98, 99 or more percent sequence identity to the protein of SEQ ID No: 1; and does not bind to factor H; and the immune response elicited is cross reactive with two or more of variant 1 factor H binding protein, variant 2 factor H binding protein and variant 3 factor H binding protein from N. meningitidis. Preferably the protein has about 95% or more sequence identity with the protein of SEQ ID No: 1.

The protection is not dependent on the serogroup but on expression of fHbp. Preferably the protection afforded by the composition of the invention will be against all N. meningitidis serogroups.

In point (ii) the mutation at position 318 may be G318D. Preferably the mutation results in a protein with reduced or no factor H binding. Preferably the binding of factor H to the modified factor H binding protein is at least 50 fold less, preferably at least two orders of magnitude less, than the binding of factor H to the wild type fHbp. Preferably the reduction in binding is measured using analyte at a concentration of about 50 nM. A reduction in binding of this order would be considered a significant reduction. Preferably this mutation results in an almost complete lack of detectable factor H binding.

Preferably, if the composition comprises the modified factor H binding protein of point (ii) it has at least 60%, 70%, 80%, 85%, 90%, 95% or more sequence identity with the sequence of FIGS. 6, 7 or 8 (Seq ID No: 2, 3 or 4).

Reference to a “cross reactive” immune response herein may mean that the immune response elicited by the isolated protein in the composition of the invention is directed to proteins other than the isolated protein used as the immunogen. In this case, a cross reactive response may mean that the immune response elicited is directed not only to the isolated protein in the immunogenic composition of the invention but also to one or more of variant 1, 2, and 3 fHbp.

The isolated protein in the composition may comprise conservative changes in the amino acid sequence, this preferably will not be taken into account when considering percent identity with SEQ ID No: 1, 2, 3 or 4. That is a conservative mutation in SEQ ID No: 1, 2, 3 or 4 may be considered when determining percent identity to be identical to the sequence of SEQ ID No: 1, 2, 3, or 4 respectively.

The isolated protein in the composition may also comprise a fragment, derivative or analog of a protein of SEQ ID No: 1, 2, 3 or 4, wherein the terms “fragment”, “derivative” and “analog” when referring to the isolated protein (SEQ ID No: 1, 2, 3 or 4), mean a protein which retains essentially the same biological function or activity as the protein of SEQ ID No: 1, 2, 3 or 4.

The fragment, derivative or analog of the isolated protein (SEQ ID NO : 1, 2, 3 or 4) may be (i) one in which one or more of the amino acid residues are substituted with a conserved or non-conserved amino acid residue (preferably a conserved amino acid residue), or (ii) one in which one or more of the amino acid residues includes a substituent group, or (iii) one in which the protein is fused with another compound, such as a compound to increase the half-life of the polypeptide (for example, polyethylene glycol), or (iv) one in which additional amino acids are fused to the protein, such as a leader or secretory sequence or a sequence which is employed for purification.

Percentage sequence identity is defined as the percentage of amino acids in a sequence that are identical with the amino acids in a provided sequence after aligning the sequences and introducing gaps if necessary to achieve the maximum percent sequence identity. Alignment for the purpose of determining percent sequence identity can be achieved in many ways that are well known to the man skilled in the art, and include, for example, using BLAST (National Center for Biotechnology Information Basic Local Alignment Search Tool).

Variations in percent identity may be due, for example, to amino acid substitutions, insertions or deletions. Amino acid substitutions may be conservative in nature, in that the substituted amino acid has similar structural and/or chemical properties, for example the substitution of leucine with isoleucine is a conservative substitution.

Preferably the immune response elicited by the composition of the invention affects the ability of N. meningitidis to infect an immunised human. Preferably the ability of N. meningitidis to infect a human immunised with the composition of the invention is impeded or prevented. This may be achieved in a number of ways. The immune response elicited may recognise and destroy N. meningitidis. Alternatively, or additionally, the immune response elicited may impede or prevent replication of N. meningitidis. Alternatively, or additionally, the immune response elicited may impede or prevent N. meningitidis causing disease in the human or non-human animal.

The isolated protein in the composition may be recombinantly produced (e.g. from a genetically-engineered expression system) or be a synthetic product, for example produced by in vitro peptide synthesis or in vitro translation.

The composition of the invention may also comprise a further one or more antigens. The further antigens may also be derived from N. meningitidis and may be capable of eliciting an immune response directed to N. meningitidis.

The composition may be used to elicit/produce a protective immune response when administered to a subject. The protective immune response may cause N. meningitidis to be killed upon infecting the subject, or it may prevent or inhibit N. meningitidis from replicating and/or from causing disease.

The composition may be used as a prophylactic or a therapeutic vaccine directed to N. meningitidis, and in particular serotype B.

According to a further aspect, the invention provides a pharmaceutical composition comprising an isolated protein with one or more of for following properties:
i) about 70, 75, 80, 85, 90, 91, 92, 93, 94, 95, 96, 97, 98, 99 or more percent sequence identity to the protein of SEQ ID No: 1;
ii) is a modified factor H binding protein, wherein the factor H binding protein has been modified at least at the position equivalent to position 318 as defined in FIG. 6 (SEQ ID No: 2);
iii) does not bind to factor H; and
iv) the immune response elicited is cross reactive with two or more of variant 1, 2 and 3 factor H binding protein from N. meningitidis;
   and a pharmaceutically acceptable carrier or excipient.

Preferably the pharmaceutical composition comprises an isolated protein with one or more of the following properties:
i) about 70, 75, 80, 85, 90, 91, 92, 93, 94, 95, 96, 97, 98, 99 or more percent sequence identity to the protein of SEQ ID No: 1;
iii) does not bind to factor H; and
iv) the immune response elicited is cross reactive with two or more of variant 1, 2 and 3 factor H binding protein from N. meningitidis;
   and a pharmaceutically acceptable carrier or excipient.

Preferably the pharmaceutical composition comprises a composition according to the first aspect of the invention, or a composition including immunogenic fragments of a protein as described with reference to the first aspect of the invention.

Preferably the pharmaceutical composition is capable of producing a protective immune response to N. meningitidis, and in particular serotype B.

The phrase “producing a protective immune response” as used herein means that the composition is capable of generating a protective response in a host organism, such as a human or a non-human mammal, to whom it is administered. Preferably a protective immune response protects against subsequent infection by N. meningitidis, and in particular serotype B. The protective immune response may eliminate or reduce the level of infection by reducing replication of N. meningitidis or by affecting the mode of action of N. meningitidis to reduce disease.

Suitable acceptable excipients and carriers will be well known to those skilled in the art. These may include solid or liquid carriers. Suitable liquid carriers include water and saline. The isolated proteins in the composition may be formulated into an emulsion or they may be formulated into biodegradable microspheres or liposomes.

The composition may further comprise an adjuvant. Suitable adjuvants will be well known to those skilled in the art, and may include Freund's Incomplete Adjuvant (for use in animals), and metal salts, such as aluminium or calcium salts.

The composition may also comprise polymers or other agents to control the consistency of the composition, and/or to control the release of the isolated protein or other antigens from the composition.

The composition may also comprise other agents such as diluents, which may include water, saline, glycerol or other suitable alcohols etc; wetting or emulsifying agents; buffering agents; thickening agents for example cellulose or cellulose derivatives; preservatives; detergents, antimicrobial agents; and the like.

Preferably the active ingredients in the composition are greater than 50% pure, usually greater than 80% pure, often greater than 90% pure and more preferably greater than 95%, 98% or 99% pure. With active ingredients approaching 100% pure, for example about 99.5% pure or about 99.9% pure, being used most often.

The composition of the present invention may be used as a vaccine against infections caused by N. meningitidis, and in particular serotype B. The composition may be used as a vaccine directed to meningitis or other invasive meningococcal diseases including septicaemia or septic shock. The vaccine may be administered prophylactically to those at risk of exposure to N. meningitidis, and/or therapeutically to persons who have already been exposed to N. meningitidis.

Preferably, if the composition is used as a vaccine, the composition comprises an immunologically effective amount of isolated protein. An “immunologically effective amount” of an isolated protein is an amount that when administered to an individual, either in a single dose or in a series of doses, is effective for treatment or prevention of infection by N. meningitidis, and in particular serotype B. This amount will vary depending upon the health and physical condition of the individual to be treated and on the isolated protein. Determination of an effective amount of an immunogenic or vaccine composition for administration to an organism is well within the capabilities of those skilled in the art.

A composition according to the invention may be for oral, systemic, parenteral, topical, mucosal, intramuscular, intravenous, intraperitoneal, intradermal, subcutaneous, intranasal, intravaginal, intrarectal, transdermal, sublingual, inhalation or aerosol administration.

The composition may be arranged to be administered as a single dose or as part of a multiple dose schedule. Multiple doses may be administered as a primary immunisation followed by one or more booster immunisations. Suitable timings between priming and boosting immunisations can be routinely determined.

Vaccine compositions may be administered in a unit dosage form of about 0.001 to 100 pg/kg (protein/body weight) and more preferably 0.01 to 10 pg/kg and most preferably 0.1 to 1 pg/kg 1 to 3 times with an interval of about 1 to 6 week intervals between immunizations.

A composition according to the invention may be used in isolation, or it may be combined with one or more other immunogenic or vaccine compositions, and/or with one or more other therapeutic regimes.

Compositions of the invention may be able to induce a serum bactericidal antibody responses and elicit antibodies which mediate opsonphagocytosis after being administered to a subject. These responses are conveniently measured in mice and the results are a standard indicator of vaccine efficacy.

The compositions of the invention may also, or alternatively, be able to elicit an immune response which neutralises bacterial proteins or other molecules, thereby preventing them from having their normal function and preventing or reducing disease progression without necessarily destroying the pathogenic organism/bacteria, in this case to N. meningitidis, and in particular serotype B.

According to a further aspect, the present invention provides the use of an isolated protein with one or more of the following properties:
i) about 70, 75, 80, 85, 90, 91, 92, 93, 94, 95, 96, 97, 98, 99 or more percent sequence identity to the protein of SEQ ID No: 1;
ii) is a modified factor H binding protein, wherein the factor H binding protein has been modified at least at the position equivalent to position 318 as defined in FIG. 6 (SEQ ID No: 2);
iii) does not bind to factor H; and
iv) the immune response elicited is cross reactive with two or more of variant 1, 2 and 3 factor H binding protein from N. meningitidis;
   in the preparation of a medicament for eliciting an immune response. The medicament may be used for the prophylactic or therapeutic vaccination of subjects against N. meningitidis, and in particular serotype B. The medicament may be a prophylactic or a therapeutic vaccine. The vaccine may be for meningitis, septicaemia and/or septic shock caused by N. meningitidis, and in particular serotype B.

According to a yet further aspect, the invention provides a composition comprising an isolated protein with one or more of the following properties:
i) about 70, 75, 80, 85, 90, 91, 92, 93, 94, 95, 96, 97, 98, 99 or more percent sequence identity to the protein of SEQ ID No: 1;
ii) is a modified factor H binding protein, wherein the factor H binding protein has been modified at least at the position equivalent to position 318 as defined in FIG. 6 (SEQ ID No: 2);
iii) does not bind to factor H; and
iv) the immune response elicited is cross reactive with two or more of variant 1, 2 and 3 factor H binding protein from N. meningitidis;
   for use in generating an immune response to N. meningitidis, and in particular serotype B. The immune response may be prophylactic or therapeutic. The composition may be for use as a vaccine.

According a still further aspect, the present invention provides a method of protecting a human or non-human animal from the effects of infection by N. meningitidis comprising administering to the human or non-human animal a composition according to any other aspect of the invention. The composition may be a vaccine.

According to another aspect, the invention provides a method for raising an immune response in a human or non-human animal comprising administering a pharmaceutical composition according to the invention to the human or non-human animal. The immune response is preferably protective. The method may raise a booster response in a patient that has already been primed. The immune response may be prophylactic or therapeutic.

One way to check the efficacy of a therapeutic treatment comprising administration of a composition according to the invention involves monitoring for N. meningitidis infection after administration of the composition. One way to check the efficacy of a prophylactic treatment comprising administration of a composition according to the invention involves monitoring immune responses to N. meningitidis after administration of the composition.

According to another aspect, the invention provides the use of an isolated protein with one or more of the following properties:
i) about 70, 75, 80, 85, 90, 91, 92, 93, 94, 95, 96, 97, 98, 99 or more percent sequence identity to the protein of SEQ ID No: 1;
ii) is a modified factor H binding protein, wherein the factor H binding protein has been modified at least at the position equivalent to position 318 as defined in FIG. 6 (SEQ ID No: 2);
iii) does not bind to factor H; and
iv) the immune response elicited is cross reactive with two or more of variant 1, 2 and 3 factor H binding protein from N. meningitidis;
   in the preparation of a medicament for use in the immunisation of human or non-human mammals against infection by N. meningitidis, and in particular serotype B.

According to a further aspect the invention provides a kit for use in inducing an immune response in an organism, comprising an immunogenic or vaccine composition according to the invention and instructions relating to administration.

In addition to their potential use as vaccines, compositions according to the invention may be useful as diagnostic reagents and as a measure of the immune competence of a vaccine.

The skilled man will appreciate that any of the preferable features discussed above can be applied to any of the aspects of the invention.

Preferred embodiments of the present invention will now be described, merely by way of example, with reference to the following figures and examples.

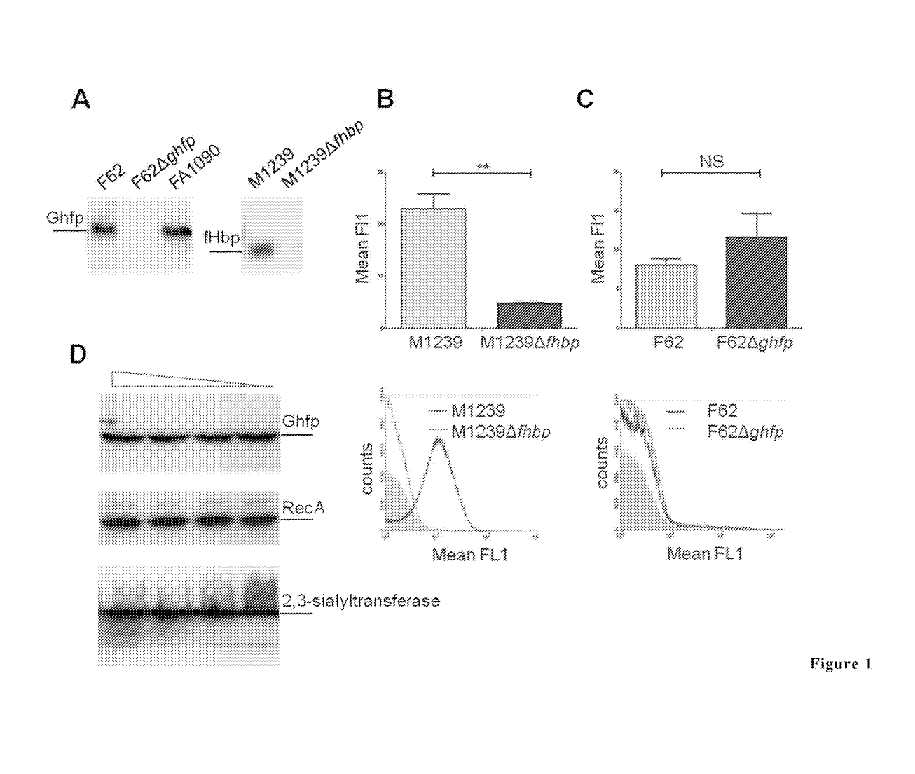

FIGS. 1a to 1d —illustrates that Ghfp is not expressed on the surface of N. gonorrhoeae. FIG. 1a shows western blot analysis of Ghfp expression by N. gonorrhoeae F62, F62Δghfp and FA1090, and fHbp V3.28 expressed by N. meningitidis strain M1239 and M1239Δfhbp. Surface expression of fHbp V3.28 (FIG. 1b) and Ghfp (FIG. 1c) was assessed by flow cytometry analysis. Error bars show the SEM of three separate experiments; **p<0.05 and NS (not significant, Student's t-test). Representative flow cytometry overlays are shown below the graphs. Bacteria incubated without anti-Ghfp sera are shown as the grey filled areas. FIG. 1d illustrates the lack of expression of Ghfp on bacteria by exposure to proteinase K (3 ng/ml and serial three-fold dilutions), the effect on proteins (shown) is determined by Western blot analysis.

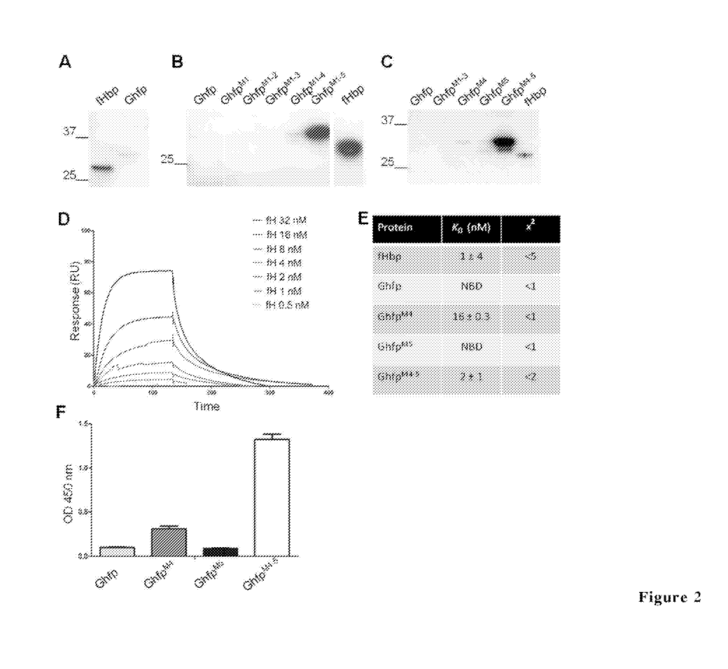

FIGS. 2a to 2f—illustrate the fH binding capacity of Ghfp. FIGS. 2a to 2c show fH binding to wild type and modified Ghfp and fHbp V3.45 assessed by far western analysis using normal human serum as the source of fH. Western blots are representatives of three separate experiments. Molecular mass is shown in kDa. FIG. 2d shows a typical equilibrium fit for binding of fH₆₋₇ to Ghfp^{M4-5}. FIG. 2e shows the results of SPR performed with Ghfp, Ghfp^{M4} (R288H), Ghfp^{M5} (D318G) and Ghfp^{M4-5} (R288H/D318G); NBD, no binding detected. FIG. 2f shows the detection of full length fH (5 nM) binding to wild-type and modified Ghfp by ELISA. Mean±SEM of three experiments are shown.

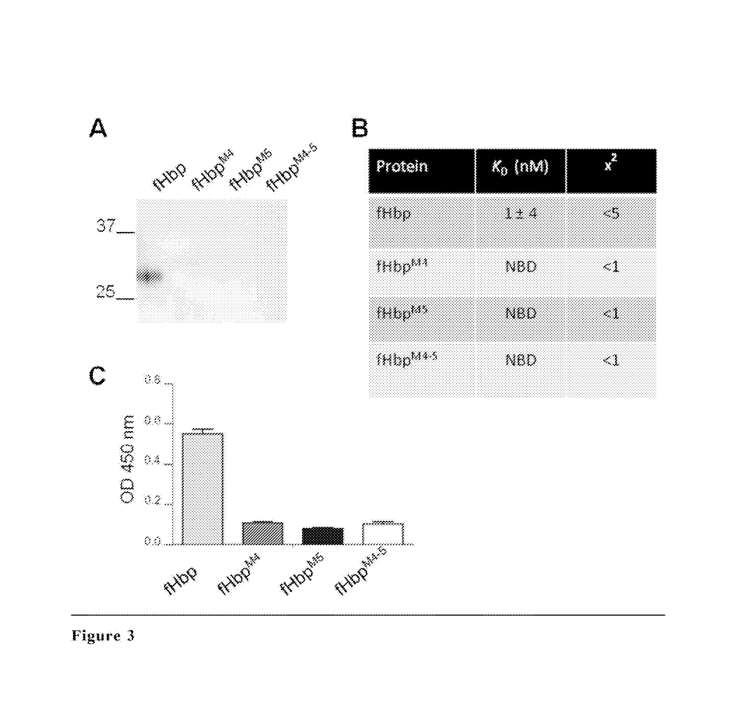

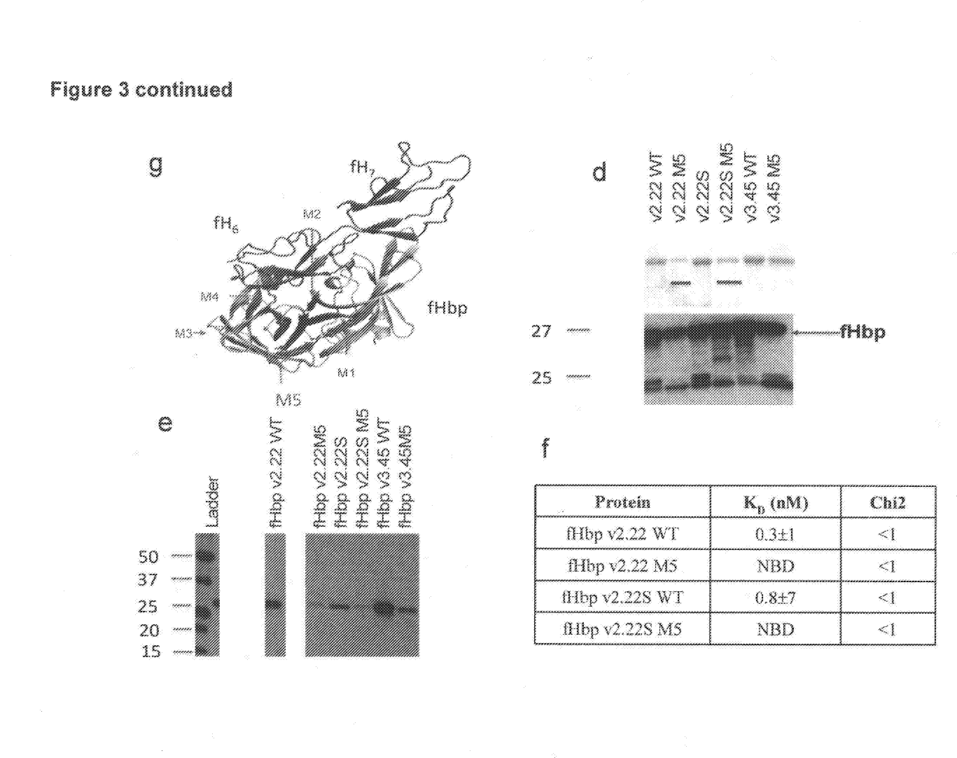

FIGS. 3a to 3c—illustrate the binding of fH to modified fHbp 3.45. FIG. 3a is an analysis of fH binding to modified V3.45 fHbp by far western using normal human serum as the source of fH. Molecular mass is shown in kDa. FIG. 3b shows SPR values of fH₆₋₇ binding to wild type and modified V3.45 fHbp; NBD, no binding detected. FIG. 3c shows the detection of full length fH (5 nM) binding to wild-type and modified V3.45 fHbp by ELISA. Data represents the mean±SEM of three different experiments.

FIGS. 3d to 3f—illustrate the binding of fH to modified fHbp 2.22 (alongside fHbp 3.45). fHbp 2.22 is a variant 2 factor H binding protein, and fHbp 3.45 is a variant 3 factor H binding protein. FIG. 3d illustrates Western blot analysis of recombinant fHbp molecules detected using anti-Ghfp serum. FIG. 3e shows fH binding to wild type and mutant fHbps assessed by far Western analysis using normal human serum. Blots are representative of 3 independent experiments. FIG. 3f shows SPR analysis showing fH6-7 binding to recombinant fHbps. NBD, no binding detected. In FIGS. 3d to 3f residue M4 is 288 and M5 is 318 (G318D). The results presented demonstrate that an amino acid mutation at position 318 in variant 2 and 3 fHbp result in reduced fH binding. V2.22S refers to a stable version of V2.22 fHbp, where stabilising mutations have been introduced remote from the fH binding site which prevent/reduce protein degradation.

FIG. 3g—is a cartoon representation of fHbp and Fh. Residues M4 and M5 are illustrated as important for fH binding.

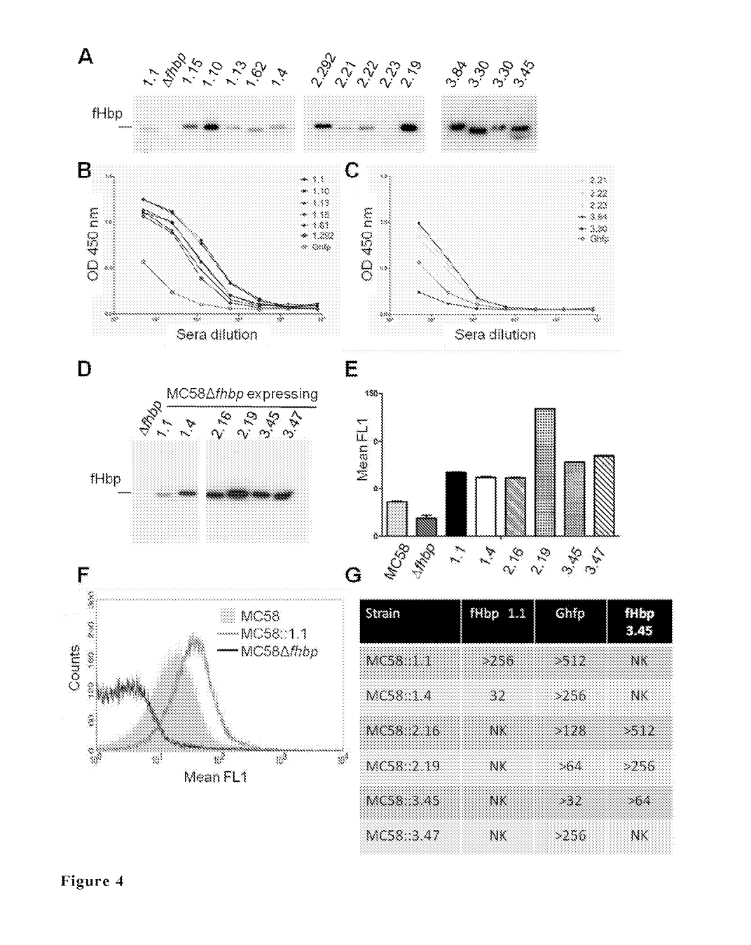

FIGS. 4a to g—illustrates the immunogenicity of Ghfp. FIG. 4d shows the detection of fHbp variants in whole cell lysates of N. meningitidis by western blot analysis using anti-Ghfp sera. FIGS. 4b and 4c shows the recognition of recombinant V1 (FIG. 4b), and V2/V3 (FIG. 4c) fHbps by anti-Ghfp sera by ELISA. FIG. 4d shows western blot analysis of whole cell lysates of fHbp expressed by isogenic MC58Δfhbp strains detected by anti-Ghfp sera. FIG. 4e shows surface expression of different fHbps in the isogenic MC58Δfhbp strains detected by flow cytometry. Graph shows the mean±SEM of three separate experiments. FIG. 4f shows representative corresponding flow cytometry overlay of MC58 (grey hatched area), MC58Δfhbp and MC58Δfhbp+fhbp V1.1 detected by anti-Ghfp by flow cytometry. FIG. 4g shows SBA responses for anti-Ghfp, fHbp V1.1, and fHbp 3.45 sera using rabbit complement; NK, no killing.

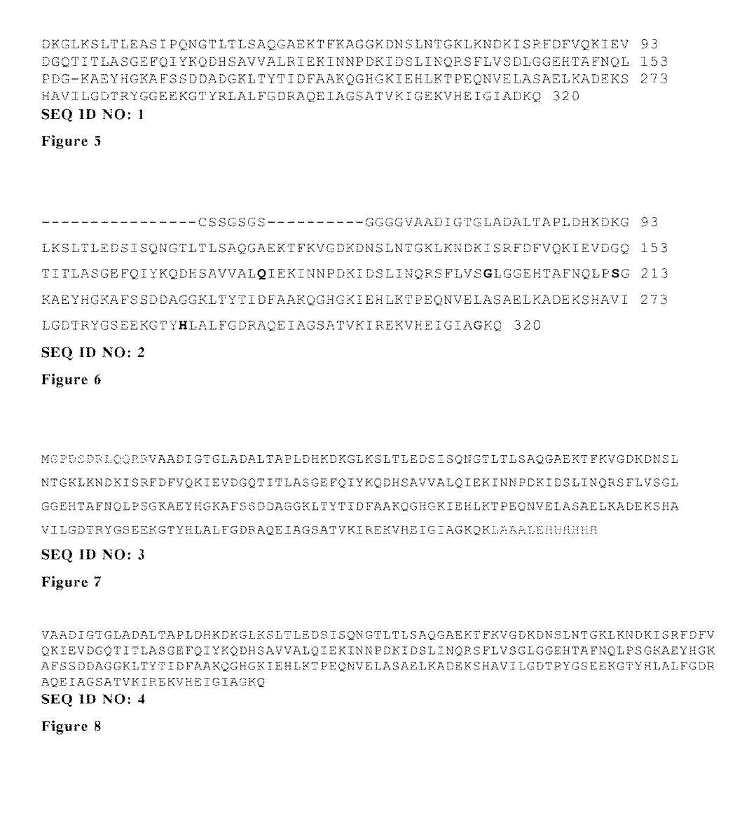

FIG. 5—is the protein sequence of Ghfp—SEQ ID No: 1

FIG. 6—is the amino acid sequence of the factor H binding protein (SEQ ID NO: 2). This protein has the GenBank Accession No: AAR84435.

FIG. 7—is a modified version of the amino acid sequence of FIG. 6, in which the C and N terminal ends have been modified for expression and purification in E. coli (SEQ ID NO: 3).

FIG. 8—is a modified version of the amino acid sequence of FIG. 6, in which the C terminal end has been modified (SEQ ID NO: 4).

### MATERIAL AND METHODS

### Bacterial Strains and Growth

The bacterial strains used herein are shown in Table 1 and Table 2. N. meningitidis was grown in the presence of 5% CO₂ at 37° C. on Brain Heart Infusion (BHI, Oxoid, Basingstoke, United Kingdom) plates with 5% (vol./vol.) horse serum (Oxoid) or in BHI broth at 37° C. N. gonorrhoeae was grown in the presence of 5% CO₂ at 37° C. on GC agar (Sigma Aldrich) plates with Vitox (Oxoid) or in GC broth (15 g Protease peptone (Oxoid), 4 g K₂HPO₄, 1 g KH₂PO₄, 5 g NaCl per litre (Sigma Aldrich) with 10 ml Kellogg's supplement (40 g glucose, 0.5 g glutamine, 50 mg Fe(NO₃)₉H₂O, 1 ml 0.2% thiamine pyrophosphate per 100 ml, Sigma Aldrich). Escherichia coli was grown on LB agar plates or LB liquid at 37° C. with appropriate antibiotics.

### Generation of Mutant Strains

Strain MC58Δfhbp [Lucidarme et al (2011) Clin Vaccine Immunol 18: 1002-1014] was complemented with pGCC4 [Mehr and Seifert (1998) Mol Microbiol 30: 697-710] containing fhbp V1.1, 1.4, 1.13, 2.16, 2.16, 3.45 and 3.47. PCR to amplify fhbp was performed using genomic DNA from strains listed in Table 1 and using primers in Table 3. PCR products were ligated into pGEMT (Promega) then pGCC4. Transformation of N. meningitidis strain MC58ΔfHbp was performed as described previously [Exley et al (2005) J Exp Med 201: 1637-1645]. M1239Δfhbp was constructed as MC58Δfhbp and F62Δghfp.

Western Blot Analysis of fHbp

N. meningitidis was grown overnight and re-suspended in phosphate buffered saline (PBS). The concentration of bacteria was determined by measuring the O.D. at 260 nm of bacterial lysates in 1% SDS/0.1M NaOH [Exley er al (2005) J Exp Med 201: 1637-1645] and adjusted to 10⁹ CFU per ml. Samples were mixed with an equal volume of 2× SDS-PAGE loading buffer and boiled for 10 minutes, then run on SDS-PAGE gels and transferred to Immobilon PVDF membranes (Millipore). Membranes were blocked with 3% skimmed milk in 0.01% Tween in PBS (PBS-T) then incubated with primary (immune sera at a 1:10000 dilution) and subsequently with secondary antibodies (goat anti-mouse conjugated HRP IgG, Dako, 1:20000 dilution) all in PBS-T with 3% skimmed milk. fH binding to fHbp expressed by N. meningitidis or recombinant proteins was analysed by far western blotting. Blots were incubated with normal human serum (diluted 1:100) for 45 minutes, then incubated with anti-fH (Quidel 1:1000 dilution), followed by rabbit anti-goat-HRP conjugated IgG (Santa Cruz 1:20000 dilution). Binding of secondary antibodies was detected using the ECL kit (Amersham).

Expression and Purification of Recombinant Ghfp and fHbp

Genes were amplified without their signal sequence by PCR with genomic DNA using primers described in Table 3. PCR products were ligated into pGEMT then into pET28a (Invitrogen, after digestion with BamHI and EcoRI) or pET21b (Invitrogen, using HindIII and XhoI, or NdeI and XhoI). Proteins were expressed in E. coli and purified using Nickel affinity chromatography followed by a HiTrapQ HP column (GE Healthcare). Mutations were introduced into ghfp by overlapping PCR and into fHbp by QuikChange Site-Directed mutagenesis (Agilent Technologies) using primers described in Table 3.

### Surface Plasmon Resonance (SPR)

SPR was performed using a Biacore 3000 (GE Healthcare). Ghfp (50 μg/ml) was first digested with 0.5 μg/ml trypsin for 2 hours at room temperature under constant shaking (300 rpm), then 0.1 mg/ml Pefabloc SC plus (Roche) added and incubated for 10 minutes prior to dialysis against PBS. Recombinant proteins were immobilized on a CM5 sensor chip (approximately 600-1000 RU) (GE Healthcare) and increasing concentrations of fH₆₋₇ (0.5 nM-32 nM) were injected over the flow channels (40 μl/min). Dissociation was allowed for 300 seconds. BIA evaluation software was used to calculate the K_{D}.

### ELISAs

Proteins (3 μg/ml, 50 μl per well) were coated on the surface of wells (F96 maxisorp, Nunc), and after blocking with 4% BSA in PBS-T, anti-Ghfp sera was added at different dilutions and detected with goat anti-mouse HRP antibody (1:5000 diluted) followed by substrate (BD). To measure fH binding to Ghfp and fHbp, proteins were coated onto wells (3 μg/ml, 50 μl per well), then incubated with fH (1 μg/ml, Sigma) and fH binding was detected using anti-fH poly clonal antibody (Quidel, 1:1000 dilution) followed by rabbit anti-goat (Dako, 1:5000 dilution).

### Generation of Anti-Ghfp Sera

Six female BALB/C mice (6-8 week old, Charles Rivers, Margate) were immunised with antigens (20 μg) absorbed to aluminium hydroxide (final concentration 3 mg/ml), 10 mM Histidine-HCL, 2M NaCl (final concentration 9 mg/ml) and distilled H₂O and mixed overnight at 4° C. The antigens were given via the intraperitioneal route on days 0, 21 and 35. Sera was collected on day 49 by terminal anaesthesia and cardiac puncture.

### Serum Bactericidal Assay (SBA)

N. meningitidis was grown on BHI plates supplemented with 1 mM IPTG overnight and suspended in PBS supplemented with 0.1% glucose (PBS-G) to a final concentration of 5×10⁴ CFU/ml. Bacteria were mixed with an equal volume of baby rabbit complement (Cedarlane) diluted 1:10 in PBS-G. Heat inactivated sera, pooled from at least six mice was added to the wells. Control wells contained either no serum or no complement. Following incubation for 1 hour at 37° C. in the presence of 5% CO₂ 10 μl from each well was plated onto BHI plates in duplicate and the number of surviving bacteria were determined. The bactericidal activity was expressed as the dilution of sera needed to kill more than 50% of bacteria in three independent experiments.

### Surface Protein Digestion

N. gonorrhoeae strain F62 was grown overnight in GC liquid at 37° C. then diluted 1:20 and grown for approximately six hours until an OD A₆₀₀ of approximately 0.5. An aliquot (1 ml) of the bacterial culture was centrifuged at 13,000×g then re-suspended in 300 μl of 3 ng/ml Proteinase K (Qiagen) or 3 times dilutions from this. After incubation for 30 minutes at 37° C., Pefablock SC inhibitor (Roche, final concentration 1 mM) was added for 15 minutes at room temperature. Samples were then spun and suspended in 100 μl 1× sample buffer. Digestion was assessed by Western blot analysis with antibodies against Ghfp (1:10000 diluted), RecA (Abcam, 1:5000 diluted), and α-2,3-Sialyltransferase [Shell D M et al (2002) Infect Immun 70: 3744-3751] (1:20000) followed by rat anti-rabbit HRP (1:20000).

### Flow Cytometry

Bacteria (1×10⁹) were fixed in 1 ml of 3% formaldehyde for two hours then washed with PBS. To measure fHbp expression, 5×10⁷ bacteria were incubated with 50 μl anti-Ghfp sera (diluted 1:500) in PBS-T for 30 minutes at 4° C. with shaking, washed in PBS-T then incubated with FITC conjugated goat anti-mouse antibody (DAKO, diluted 1:50) for 30 minutes. After washing, fHbp expression was measured by flow cytometry using the FACS calibur, calculating the mean FL1 of 10000 bacteria.

TABLE 1
N. meningitidis and N. gonorrhoeae isolates

| | fHbp | | | | | |
|---|---|---|---|---|---|---|
| Strain | allele | ST | CC | Year | Group | REF |
| N. meningitidis | | | | | | |
| MC58 | 1.1 | 74 | 32 | 1983 | B | |
| MC58Δfhbp | | | | | | Lucidarme, |
| | | | | | | 2011 |
| H44/76 | 1.1 | 32 | 32 | 1976 | B | |
| M1239 | 3.28 | 437 | 41/44 | 1994 | | |
| m1239Δfhbp | | | | | | This study |
| BZ10 | 2.16 | | | | | |
| fam 18 | 2.22 | | | | | |
| M07 0240624 | 1.P13 | 6781 | N/A | 2007 | B | |
| M01 240101 | 1.P15 | 1049 | cc269 | 2001 | B | |
| M07 0240606 | 3.P45 | 213 | cc213 | 2007 | B | |
| M07 0240625 | 2.P21 | 1466 | cc174 | 2007 | Y | |
| M07 0240686 | 2.P22 | 7779 | cc11 | 2007 | C | |
| M07 0240877 | 3.P30 | 1214 | cc269 | 2007 | B | |
| M07 0240684 | 2.P25 | 1655 | cc23 | 2007 | Y | |
| M08 0240023 | 3.P84 | 6788 | cc41/44 | 2008 | B | |
| M07 0240688 | 3.P47 | 1946 | cc461 | 2007 | B | |
| M07 0240662 | 1.P276 | 213 | cc213 | 2007 | B | |
| M08 0240533 | 1.P254 | 6602 | cc269 | 2008 | B | |
| M07 0240615 | 1.P37 | 18 | cc18 | 2007 | B | |
| M07 0240839 | 1.P61 | 1163 | cc269 | 2007 | B | |
| M07 0240675 | 1.292 | 269 | cc269 | 2007 | B | |
| M07 0241034 | 1.P249 | 461 | cc461 | 2007 | B | |
| M01 241133 | 1.P9 | 11 | cc11 | 2001 | W135 | |
| M01 240185 | 1.P10 | 4 | cc11 | 2001 | B | |
| 92053 | 1.P5 | 656 | cc5 | 1992 | A | |
| 2005167 | 2.P23 | 181 | 181 | 2005 | X | |
| M00.242922 | 1.4 | | | | | |
| M01.240013 | 2.19 | | | | | |
| N. | | | | | | |
| gonorrhoeae | | | | | | |
| F62 | | | | 1900 | | |
| F62Δghfp | | | | | | |
| FA1090 | | | | 1899 | | |

TABLE 2
E. coli strains used in this study

| E. coli | | | | |
|---|---|---|---|---|
| Strain | Plasmid | Gene | Mutation | Reference |
| BL21 | pET28b | ghfp | | This Study |
| BL21 | pET28b | ghfp M1 | R141Q | This Study |
| BL21 | pET28b | ghfp M1-2 | R141Q, D164G | This Study |
| BL21 | pET28b | ghfp M1-3 | R141Q, D164G, | This Study |
| | | | D177S | |
| BL21 | pET28b | ghfp M1-4 | R141Q, D164G, | This Study |
| | | | D177S, R253H | |
| BL21 | pET28b | ghfp M1-5 | R141Q, D164G, | This Study |
| | | | D177S, R253H, | |
| | | | D283G | |
| BL21 | pET28b | ghfp M4 | R253H | This Study |
| BL21 | pET28b | ghfp M5 | D283G | This Study |
| BL21 | pET28b | ghfp M4-5 | R253H, D283G | This Study |
| BL21 | pET21b | fHbp 3.45 | | This Study |
| BL21 | pET21b | fHbp 3.45 M4 | H222R | This Study |
| BL21 | pET21b | fHbp 3.45 M5 | G318D | This Study |
| BL21 | pET21b | fHbp 3.45 M4-5 | H222R, G318D | This Study |

TABLE 3
primers used in this study

| Primer Name | Sequence |
|---|---|
| ghfp F | GCGGATCCATGACTAGGAGTAAAC |
| ghfp R | GCGAATTCCTACTGTTTGTCGGCG |
| fHbp V3.45 F | CGCGGATCCCATATGAGCAGCGGAAGCGGAAGC |
| fHbp V3.45 R | GCCCAAGCTTCTGTTTGCCGGCGATGCC |
| ghfp M1F | GGT GCC CTA CAG ATT GAA AAA |
| ghfp M1R | TTT TTC AAT CTG TAG GGC AAC |
| ghfp M2F | CTTGTCAGCGGCTTGGGCGGA |
| ghfp M2R | TCCGCCCAAGCCGCTGACAAG |
| ghfp M3F | CAACTGCCTGGCGGCAAAGCC |
| ghfp M3R | GGCTTTGCCGCCAGGCAGTTG |
| ghfp M4F | GAAGAGAAAGGCACTTACCACCTCGCCCTTTTC |
| | GGCGAC |
| ghfp M4R | GTCGCCGAAAAGGGCGAGGTGGTAAGTGCCTTT |
| | CTCTTC |
| ghfp M5 R | GCGAATTCCTACTGTTTGCCGGCG |
| fHbp 3.45 M4F | GAAAAAGGCACTTACCGCCTCGCTCTTTTCGGC |
| fHbp 3.45 M4R | CTTTTTCCGTGAATGGCGGAGCGAGAAAAGCCG |
| fHbp 3.45 M5F | GAAATCGGCATCGCCGACAAACAGAAGCTTGCG |
| fHbp 3.45 M5R | CTTTAGCCGTAGCGGCTGTTTGTCTTCGAACAC |
| pGCC4V1.1F | CGGTTAATTAAGGAGTAATTTTTGTGAATCGAA |
| | CTGCCTTCTGCT |
| pGCC4V1.4F | CGGTTAATTAAGGAGTAATTTTTGTGAACCGAA |
| | CTGCC |
| pGCC4V1.15F | CGGTTAATTAAGGAGTAATTTTTGTGAACCGAA |
| | CTACC |
| pGCC4V2.16F | CGGTTAATTAAGGAGTAATTTTTGTGAACCGAA |
| | CTGCCTTCTGCT |
| pGCC4V3.47F | CGG TTAATTAAGGAGTAATTTTTGTGAACCGA |
| | ACTACC |
| pGCC4V1.1R | CGGTTAATTAATTATTGCTTGGCGGC |
| pGCC4V1.4R | CGGTTAATTAATTACTGCTTGGCGGCAAGAC |
| PGCC4V1.15R | CGGTTAATTAATTATTGCTTGGCGGCAAGAC |
| PGCC4V2.16R | CGGTTAATTAACTACTGTTTGCCGGCGATGC |
| fHbp 1.10 F | CGCGGATCCCATATGGTTGCCGCCGACATCG |
| fHbp 1.10 R | CCCGCTCGAGCTGCTTGGCGGCAAGAC |
| fHbp 1.13 F | CCCGCTCGAGCTGCTTGGCGGCAAGAC |
| fHbp 1.13 R | CCCGCTCGAGCTGCTTGGCGGCAAGAC |
| fHbp 1.15 F | CGCGGATCCCATATGGTCGCCGCCGACATCG |
| fHbp 1.15 R | CCCGCTCGAGTTGCTTGGCGGCAAGAC |
| fHbp 1.61 F | CGCGGATCCCATATGGTCGCCGCCGACATTG |
| fHbp 1.61 R | CCCGCTCGAGTTGCTTGGCGGCAAGAC |
| fHbp 1.229 F | CGCGGATCCCATATGGTCGCCGCCGACATCG |
| fHbp 1292 R | CCCGCTCGAGCTGCTTGGCGGCAAGAC |
| fHbp V2 and V3 F | CGCGGATCCCATATGGGCCCTGATTCTGACCGC |
| | CTGCAGCAGCGGAGGGTCGCCGCCGACATCGG |
| fHbp V2 R | CCCGCTCGAGCTGTTTGCCGGCGATGCC |
| fHbp V3 R | GCCCAAGCTTCTGTTTGCCGGCGATGCT |

### Results

### Ghfp is Not Surface Expressed

Analysis of the genome sequence of N. gonorrhoeae strain FA1090 identified the presence of a fHbp homologue [Hadad et al (2012) APMIS 120: 750-760 and Welsch and Ram (2008) Vaccine 26 Suppl 8: 140-145] which was designated Gonococcal homologue of fHbp, Ghfp. Sequence alignment of Ghfp with the available fHbp sequences (www.neisseria.org) reveals that Ghfp has between 60-67%, 81-89% and 86-94% amino acid identity with V1, V2 and V3 fHbp, respectively (Supplementary FIG. 1). To investigate the cellular location of Ghfp, anti-sera were raised against recombinant Ghfp from N. gonorrhoeae strain FA1090. By Western blot analysis, sera recognised a protein with an estimated molecular weight of 30 kDa (corresponding to Ghfp) in lysates of N. gonorrhoeae; no protein was detected in lysates from the Ghfp mutant (FIG. 1A). Sera raised against Ghfp also recognise V3.28 fHbp expressed by N. meningitidis strain M1239 (FIG. 1A). To determine whether Ghfp is surface located, flow cytometry analysis was performed with anti-Ghfp sera to detect Ghfp on the surface of N. gonorrhoeae F62 and F62Δghfp, and fHbp on N. meningitidis M1239 and M1239Δfhbp (FIGS. 1B and C). Results demonstrate that anti-Ghfp sera recognise V3.28 fHbp on the surface of N. meningitidis, but there was no detectable Ghfp on the gonococcus.

To exclude the possibility that the lack of detection of Ghfp by flow cytometry was due to low expression levels, viable bacteria were exposed to proteinase K, and the degradation of Ghfp, a surface protein i.e. the α-2,3-sialyltransferase [Shell et el (2002) Infect Immun 70: 3744-3751], and the cytoplasmic protein RecA was monitored by western blot analysis. Ghfp and RecA were unaffected by exposing cells to proteinase K, while there was degradation of the α-2,3-sialyltransferase (FIG. 1D). Recombinant Ghfp is cleaved by these concentrations of proteinase K (data not shown) demonstrating that Ghfp is susceptible to cleavage by this protease. In conclusion, the results demonstrate that Ghfp is expressed by N. gonorrhoeae but is not located on the bacterial surface, in keeping with previous predictions.

### Identification of Residues Responsible for Low Affinity Binding of Ghfp to fH

Due to its high sequence identity with V3 fHbp, which binds fH with a K_{D} in the nM range [Johnson et al (2012) PLoS Pathog 8: e1002981 and Seib et al (2011) Infect Immun 79: 970-981], fH binding to Ghfp was tested by far western analysis. Surprisingly, there was no detectable fH binding to Ghfp using normal human serum as the source of fH (FIG. 2A). Therefore, sequence of Ghfp was compared with V2 and V3 fHbps that bind fH at high affinity, and five amino acids were identified that are consistently different between Ghfp and V2/V3 fHbps i.e. R176, D199, D212, R288 and D318 of Ghfp (amino acid numbering according to fHbp V1.1 structure [Schneider et al (2009) Nature 458: 890-893]. The amino acids R176 and D199 are located in the predicted N-terminal β barrel of Ghfp and, similar to C-terminal β barrel residue D212, are not located at the fH:Ghfp interface. In contrast, R288 is located in close proximity to the predicted fH:Ghfp interface, while D318 could be involved in interactions between the two predicted β barrels of Ghfp. To determine whether these five amino acid changes are responsible for the reduced fH binding to Ghfp, the specific amino acids were modified into the equivalent residues in the closely related V3.45 fHbp. Modification of these five residues in Ghfp (i.e. R176Q, D199G, D212S, R288H and D318G) was sufficient to enable Ghfp to bind fH by far western analysis (FIG. 2B). Analysis of recombinant Ghfp with these changes either singly or in combination demonstrated that the substitutions, R288H and D318G, are sufficient to confer fH binding to Ghfp by far western analysis (FIG. 2C). To further analyse this interaction in more detail, the binding of Ghfp and V3.45 fHbp to fH₆₋₇ was also investigated by Surface Plasmon Resonance (SPR, FIG. 2D). The dissociation constant of V3.45 fHbp and fH was 1±4 nM, similar to previous results for V3 fHbps [12,13]. Consistent with the far western analysis, no fH binding was detected to Ghfp by SPR under these conditions. Moreover, no fH binding was observed to Ghfp^{M5} (D318G). There was fH binding detected to Ghfp^{M4} (R288H) (K_{D} of 16±0.3 nM) while the double substitution, Ghfp^{M4-5} (R288H/D318G), resulted in fH binding that was equivalent to fHbp (K_{D} i.e. of 2 nM).

To exclude the possibility that Ghfp interacts with fH via CCP domains other than fH₆₋₇ fH binding was also examined by ELISA in which recombinant proteins were coated on the wells of plates and binding to purified full length fH was detected. Consistent with SPR, fH binding to Ghfp^{M4-5}, partial fH binding to Ghfp^{M4}, and no fH binding to wild-type Ghfp or Ghfp^{M5} was observed (FIG. 2E). In conclusion, despite its high amino acid identity with V3 fHbp, Ghfp does not bind fH to any significant degree, and there are only two amino acids responsible for the striking difference in affinity compared with fHbp.

fH Binding to Modified fHbp V3.45

As modification of R288H and D318G in Ghfp confers high affinity fH binding, it was considered whether the corresponding residues in V3.45 fHbp are necessary for binding to fH. V3.45 fHbp with H288R and G318D (fHbp^{M4-5}) was generated. Initially binding of fH was examined by far Western analysis (FIG. 3A) and showed loss of detectable fH binding to fHbp^{M4} (H288R), fHbp^{M5} (G318D) and fHbp^{M4-5}. To verify these results, binding of fH₆₋₇ to fHbp wild type and modified proteins was analysed by SPR (FIG. 3B). No detectable binding of fH was observed to fHbp^{M4}, fHbp^{M5} and fHbp^{M4-5} under these conditions, demonstrating that both of these residues is necessary for high affinity interactions with fH. To exclude the possibility that these modified fHbp molecules interact with fH via CCP domains other than fH₆₋₇, we examined binding to purified full length fH by ELISA. Consistent with SPR, we observed fH binding to fHbp V3.45 but no binding to fHbp^{M4}, fHbp^{M5} and fHbp^{M4-5} (FIG. 3C). Taken together, it may be concluded that the amino acids present at positions 288 and 318 are the basis for the profound difference in interactions with fH observed in the closely related proteins from the gonococcus and meningococcus.

Similar results were obtained with a variant 2 fHbp, V2.22, as illustrated in FIGS. 3d to 3f.

### Immunogenicity of Ghfp

The vaccine potential of Ghfp was demonstrated by examining the ability of sera raised against this protein to recognise fHbps expressed by a range of N. meningitidis isolates. Immune sera not only recognises closely related V3 fHbps expressed in whole cell extracts of N. meningitidis but also V1 and V2 proteins (FIG. 4A). Surprisingly, immune sera raised against Ghfp detected all V1, V2 and V3 fHbps examined (FIGS. 4B and C).

To determine whether immunisation with Ghfp elicits functional immune responses, isogenic strains of N. meningitidis MC58 were constructed each expressing one of the seven most prevalent fHbps (i.e. V1.1, V1.4, V2.16, V2.19, V3.45 and V3.47) from disease isolates in England and Wales, accounting for 70% [Lucidarme J et al (2010) Clin Vaccine Immunol 17: 919-929]. The wild-type copy of fHbp was inactivated and a single copy of the gene encoding each of the selected variants was introduced at an ectopic site under the control of an IPTG inducible promoter. Expression of the different fHbps was confirmed by Western blot analysis of whole cell extracts (FIG. 4D) and surface expression verified by flow cytometry (FIGS. 4E and F). Serum bactericidal activity (SBA) is an established correlate of protective immunity against meningococcal infection. The SBA of anti-Ghfp sera against the isogenic N. meningitidis strains was determined and compared with the findings with sera raised against V1.1 or V3.45 fHbp (FIG. 4G). The results show that anti-Ghfp sera exhibited SBA against N. meningitidis expressing V1.1, V1.4, V2.21, V2.22, V3.45 and V3.47. In contrast, anti-fHbp V1.1 sera only elicited SBA responses against V1.1 and V1.4 expressing strains, while anti-V3.45 fHbp sera had SBA against all V2 as well as the V3.45 expressing strains. No detectable SBA was measured with any sera against the isogenic MC58Δfhbp strain or using sera from mice receiving adjuvant alone (data not shown). In conclusion, Ghfp elicits SBA against V1, V2 and V3 fHbp expressing N. meningitidis and is therefore a naturally occurring protein capable of providing cross-protection of this nature.

### Discussion

N. meningitidis and N. gonorrhoeae are two human specific, closely related pathogens that inhabit distinct niches in the body. N. gonorrhoeae causes sexually transmitted infections predominantly affecting the mucous membranes of the genito-urinary tract, while N. meningitidis colonises the nasopharynx . Despite sharing many similarities of the genetic level, these bacteria employ entirely different mechanisms to evade immune responses, and in particular, to avoid complement activation on their surface. For example, disease isolates of N. meningitidis express a polysaccharide capsule which is essential for high-level serum resistance, while N. gonorrhoeae is not encapsulated. Instead sialylation of lipopolysaccharide markedly promotes complement resistance in the gonococcus, but this has substantially less impact on N. meningitidis.

Both organisms have evolved to bind fH to their surface to prevent complement activation (by down-regulating the alternative pathway) but use distinct strategies. The gonococcus recruits fH via an exposed surface loop of Por1A (loop 5), an outer membrane porin often expressed by isolates recovered from patients. fH can also bind to gonococci expressing Por1B albeit to a lesser degree, with this interaction facilitated by lipopolysaccharide sialylation. Although meningococci express class 3 and class 2 porins (which are related to Por1A and Por1B of N. gonorrhoeae, respectively), these are not involved in fH binding; loop 5 of the meningococcal porins lacks a region present in gonococcal Por1A, which probably accounts for its inability to bind fH. Instead, the surface expressed lipoprotein fHbp mediates high affinity binding of fH by the meningococcus irrespective of variant group. This interaction enhances bacterial survival in whole blood and prevents serum dependent killing. It is not clear why the organisms have adopted these alternative approaches to exploit the same molecule, but it is likely to be influenced by the affinity of the interaction, the local availability of fH and the density of the bacterial receptor, as well as other factors conferring complement resistance. Without capsules, gonococci are largely reliant on their capacity to recruit fH and C4bp to survive in the human host. Therefore the relatively low levels of fH in the genito-urinary tract may have favoured its recruitment by a highly abundant protein on the gonococcal surface, such as porin.

The data presented herein demonstrates that Ghfp, the gonococcal homologue of the meningococcal fH receptor, does not bind fH to any detectable extent despite its high sequence identity with fHbp. Remarkably, only two amino acids in Ghfp that differ from those in fHbp are responsible for this lack of interaction. Furthermore, the replacement of the equivalent amino acids in V3.45 fHbp (i.e. H288R and G318D) resulted in loss of fH binding. The H288R modification is located at the fH:fHbp interface; the His side of fHbp H288 sits in a hydrophobic pocket in fH formed by H337, Y353 and the methylene groups of the R341. The extended side chain of Ghfp R288 is too long to fit into this pocket without remodelling the interface, and would also result in electrostatic repulsion with R341 of fH. The lack of fH binding to V3.45 fHbp^{M5} (i.e. G318D) is more difficult to explain as it is located away from the fH:fHbp interface, and is at the end of the final strand of the second β barrel. However, the register of this strand is such that the side chain of residue 318 points into the hydrophobic core of the barrel. Substitution of Gly with Asp is not possible without structural rearrangement due to steric clashes in the hydrophobic core as it is energetically unfavourable to place a negative charge in the hydrophobic environment. Given this final strand also makes crucial contacts with the first β barrel, this substitution could lead to structural rearrangements at interface between the two barrels and therefore alter the distal fH binding site (which comprises both barrels). Recently, several residues were identified in V1, V2 and V3 fHbps which are needed for high affinity interactions with fH through alanine scanning mutagenesis [Johnson et al (2012) PLoS Pathog 8: e1002981]. Here two further mutations are taught that abolish fH:fHbp binding by analysing the binding characteristics of a natural protein.

While fHbp is located on the surface of the meningococcus, the data herein demonstrates that Ghfp is not on the external surface of the gonococcus. Closer examination of Ghfp also reveals the absence of a signal sequence for export so the protein is likely to remain intracellular and is not secreted into the extracellular milieu. The function of Ghfp remains unknown.

fHbp is a key component of protein sub-unit meningococcal vaccines under late phase clinical development. Unfortunately, antibody responses against fHbp are thought to be largely variant specific. Therefore fHbp-based vaccines consisting of a single natural fHbp might be expected to have limited coverage. To overcome this issue, vaccines under development have included fHbp together with other antigens namely GNA2132, NadA, GNA1030 and GNA2091 and PorA or multiple fHbp variants. The data presented herein shows that anti-sera raised against Ghfp has the potential to recognize representative V1, V2 and V3 fHbps, in contrast to sera raised against the widely used V1.1 fHbp and V3.45 fHbp. More importantly, the data shows that Ghfp has the potential to elicit SBA against isogenic strains expressing the most common V1, V2 and V3 fHbps in MenB.

In summary, Ghfp is a promising vaccine candidate against N. meningitidis since the protein not only offers a broad range of protection, but is also a naturally occurring non-fH binding molecule. There are potential drawbacks for the use of functional fHbps as a vaccine antigen due to its high affinity binding with fH. The extensive binding of fH to fHbp could shield immunogenic epitopes on the antigen resulting in less effective antibody responses. Moreover, binding of fHbp to fH might reduce the immunogenicity at the site where antibody responses are initiated or it could lead to formation of anti fH responses in the human host.

1. An immunogenic composition capable of eliciting an immune response when administered to a human or non-human animal, wherein the composition comprises an isolated protein with one or more of the following properties:
i) about 70, 75, 80, 85, 90, 91, 92, 93, 94, 95, 96, 97, 98, 99 or more percent sequence identity to the protein of SEQ ID No: 1 or a fragment, derivative or analog thereof;
ii) is a modified factor H binding protein, wherein the factor H binding protein has been modified at least at the position equivalent to position 318 as defined in FIG. 6 (SEQ ID No: 2);
iii) does not bind to factor H; and
iv) the immune response elicited is cross reactive with two or more of variant 1 factor H binding protein, variant 2 factor H binding protein and variant 3 factor H binding protein from N. meningitidis.

2. The immunogenic composition of claim 1 wherein the isolated protein has about 95% or more sequence identity with the protein of SEQ ID No: 1.

3. The immunogenic composition of claim 1 wherein the isolated protein has about 95% or more sequence identity with the protein of SEQ ID No: 1, and wherein the isolated protein in the composition does not bind to factor H, and wherein the immune response elicited by the composition is cross reactive to variant 1, 2 and 3 factor H binding proteins from N. meningitidis.

4. The immunogenic composition of claim 1 wherein in point (ii) the mutation at position 318 may be G318D.

5. The immunogenic composition of claim 4 wherein the mutation results in a protein with reduced or no factor H binding.

6. The immunogenic composition of claim 4 or 5 wherein composition comprises at least 60%, 70%, 80%, 85%, 90%, 95% or more sequence identity with the sequence of FIG. 6, 7 or 8 (SEQ ID No: 2, 3 or 4).

7. The immunogenic composition of any preceding claim further comprising a further one or more additional antigens.

8. Use of a composition of any preceding claim to elicit/produce a protective immune response when administered to a subject.

9. A composition of any preceding claim for use in eliciting/producing a protective immune response in a subject.

10. The use of claim 8 or 9 wherein the composition may be used as a prophylactic or a therapeutic vaccine directed to N. meningitidis, and in particular serotype B.

11. A pharmaceutical composition comprising an isolated protein with one or more of for following properties:
i) about 70, 75, 80, 85, 90, 91, 92, 93, 94, 95, 96, 97, 98, 99 or more percent sequence identity to the protein of SEQ ID No: 1 or a fragment, derivative or analog thereof;
ii) is a modified factor H binding protein, wherein the factor H binding protein has been modified at least at the position equivalent to position 318 as defined in FIG. 6 (SEQ ID No: 2);
iii) does not bind to factor H; and
iv) the immune response elicited is cross reactive with two or more of variant 1, 2 and 3 factor H binding protein from N. meningitidis;
and a pharmaceutically acceptable carrier or excipient.

12. The pharmaceutical composition of claim 11 comprising an isolated protein with the following properties:
i) about 70, 75, 80, 85, 90, 91, 92, 93, 94, 95, 96, 97, 98, 99 or more percent sequence identity to the protein of SEQ ID No: 1 or a fragment, derivative or analog thereof;
iii) does not bind to factor H; and
iv) the immune response elicited is cross reactive with two or more of variant 1, 2 and 3 factor H binding protein from N. meningitidis;
and a pharmaceutically acceptable carrier or excipient.

13. A vaccine against infections caused by N. meningitidis comprising a composition according to any preceding claim.

14. The vaccine of claim 13 wherein the vaccine comprises an immunologically effective amount of isolated protein.

15. The use of an isolated protein with one or more of the following properties:
i) about 70, 75, 80, 85, 90, 91, 92, 93, 94, 95, 96, 97, 98, 99 or more percent sequence identity to the protein of SEQ ID No: 1;
ii) is a modified factor H binding protein, wherein the factor H binding protein has been modified at least at the position equivalent to position 318 as defined in FIG. 6 (SEQ ID No: 2);
iii) does not bind to factor H; and
iv) the immune response elicited is cross reactive with two or more of variant 1, 2 and 3 factor H binding protein from N. meningitidis;
in the preparation of a medicament for eliciting an immune response.

16. A method of protecting a human or non-human animal from the effects of infection by N. meningitidis comprising administering to the human or non-human animal a composition according to any preceding claim.

17. A method for raising an immune response in a human or non-human animal comprising administering a pharmaceutical composition according to the invention to the human or non-human animal.

18. A kit for use in inducing an immune response in an organism, comprising an immunogenic or vaccine composition according to the invention and instructions relating to administration.


SEQUENCE LISTING (43 sequences)

SEQ ID NO: 1 - protein, 230 aa, Neisseria gonorrhoeae
DKGLKSLTLEASIPQNGTLTLSAQGAEKTFKAGGKDNSLNTGKLKNDKISRFDFVQKIEVDGQTITLASGEFQIYKQDHSAVVALRIEKINNPDKIDSLINQRSFLVSDLGGEHTAFNQLPDGKAEYHGKAFSSDDADGKLTYTIDFAAKQGHGKIEHLKTPEQNVELASAELKADEKSHAVILGDTRYGGEEKGTYRLALFGDRAQEIAGSATVKIGEKVHEIGIADKQ

SEQ ID NO: 2 - protein, 261 aa, Neisseria meningitidis
CSSGSGSGGGGVAADIGTGLADALTAPLDHKDKGLKSLTLEDSISQNGTLTLSAQGAEKTFKVGDKDNSLNTGKLKNDKISRFDFVQKIEVDGQTITLASGEFQIYKQDHSAVVALQIEKINNPDKIDSLINQRSFLVSGLGGEHTAFNQLPSGKAEYHGKAFSSDDAGGKLTYTIDFAAKQGHGKIEHLKTPEQNVELASAELKADEKSHAVILGDTRYGSEEKGTYHLALFGDRAQEIAGSATVKIREKVHEIGIAGKQ

SEQ ID NO: 3 - protein, 275 aa, Neisseria meningitidis
MGPDSDRLQQRRVAADIGTGLADALTAPLDHKDKGLKSLTLEDSISQNGTLTLSAQGAEKTFKVGDKDNSLNTGKLKNDKISRFDFVQKIEVDGQTITLASGEFQIYKQDHSAVVALQIEKINNPDKIDSLINQRSFLVSGLGGEHTAFNQLPSGKAEYHGKAFSSDDAGGKLTYTIDFAAKQGHGKIEHLKTPEQNVELASAELKADEKSHAVILGDTRYGSEEKGTYHLALFGDRAQEIAGSATVKIREKVHEIGIAGKQKLAAALEHHHHHH

SEQ ID NO: 4 - protein, 250 aa, Neisseria meningitidis
VAADIGTGLADALTAPLDHKDKGLKSLTLEDSISQNGTLTLSAQGAEKTFKVGDKDNSLNTGKLKNDKISRFDFVQKIEVDGQTITLASGEFQIYKQDHSAVVALQIEKINNPDKIDSLINQRSFLVSGLGGEHTAFNQLPSGKAEYHGKAFSSDDAGGKLTYTIDFAAKQGHGKIEHLKTPEQNVELASAELKADEKSHAVILGDTRYGSEEKGTYHLALFGDRAQEIAGSATVKIREKVHEIGIAGKQ

SEQ ID NO: 5 - DNA, 24 bp, Artificial Sequence
  primer
gcggatccatgactaggagtaaac

SEQ ID NO: 6 - DNA, 24 bp, Artificial Sequence
  Primer
gcgaattcctactgtttgtcggcg

SEQ ID NO: 7 - DNA, 33 bp, Artificial Sequence
  Primer
cgcggatcccatatgagcagcggaagcggaagc

SEQ ID NO: 8 - DNA, 28 bp, Artificial Sequence
  Primer
gcccaagcttctgtttgccggcgatgcc

SEQ ID NO: 9 - DNA, 21 bp, Artificial Sequence
  Primer
ggtgccctacagattgaaaaa

SEQ ID NO: 10 - DNA, 21 bp, Artificial Sequence
  Primer
tttttcaatctgtagggcaac

SEQ ID NO: 11 - DNA, 21 bp, Artificial Sequence
  Primer
cttgtcagcggcttgggcgga

SEQ ID NO: 12 - DNA, 21 bp, Artificial Sequence
  Primer
tccgcccaagccgctgacaag

SEQ ID NO: 13 - DNA, 21 bp, Artificial Sequence
  Primer
caactgcctggcggcaaagcc

SEQ ID NO: 14 - DNA, 21 bp, Artificial Sequence
  Primer
ggctttgccgccaggcagttg

SEQ ID NO: 15 - DNA, 39 bp, Artificial Sequence
  Primer
gaagagaaaggcacttaccacctcgcccttttcggcgac

SEQ ID NO: 16 - DNA, 39 bp, Artificial Sequence
  Primer
gtcgccgaaaagggcgaggtggtaagtgcctttctcttc

SEQ ID NO: 17 - DNA, 24 bp, Artificial Sequence
  Primer
gcgaattcctactgtttgccggcg

SEQ ID NO: 18 - DNA, 33 bp, Artificial Sequence
  Primer
gaaaaaggcacttaccgcctcgctcttttcggc

SEQ ID NO: 19 - DNA, 33 bp, Artificial Sequence
  Primer
ctttttccgtgaatggcggagcgagaaaagccg

SEQ ID NO: 20 - DNA, 33 bp, Artificial Sequence
  Primer
gaaatcggcatcgccgacaaacagaagcttgcg

SEQ ID NO: 21 - DNA, 33 bp, Artificial Sequence
  Primer
ctttagccgtagcggctgtttgtcttcgaacac

SEQ ID NO: 22 - DNA, 45 bp, Artificial Sequence
  Primer
cggttaattaaggagtaatttttgtgaatcgaactgccttctgct

SEQ ID NO: 23 - DNA, 38 bp, Artificial Sequence
  Primer
cggttaattaaggagtaatttttgtgaaccgaactgcc

SEQ ID NO: 24 - DNA, 38 bp, Artificial Sequence
  Primer
cggttaattaaggagtaatttttgtgaaccgaactacc

SEQ ID NO: 25 - DNA, 45 bp, Artificial Sequence
  Primer
cggttaattaaggagtaatttttgtgaaccgaactgccttctgct

SEQ ID NO: 26 - DNA, 38 bp, Artificial Sequence; identical to SEQ ID NO: 24
  Primer

SEQ ID NO: 27 - DNA, 26 bp, Artificial Sequence
  Primer
cggttaattaattattgcttggcggc

SEQ ID NO: 28 - DNA, 31 bp, Artificial Sequence
  Primer
cggttaattaattactgcttggcggcaagac

SEQ ID NO: 29 - DNA, 31 bp, Artificial Sequence
  Primer
cggttaattaattattgcttggcggcaagac

SEQ ID NO: 30 - DNA, 31 bp, Artificial Sequence
  Primer
cggttaattaactactgtttgccggcgatgc

SEQ ID NO: 31 - DNA, 31 bp, Artificial Sequence
  Primer
cgcggatcccatatggttgccgccgacatcg

SEQ ID NO: 32 - DNA, 27 bp, Artificial Sequence
  Primer
cccgctcgagctgcttggcggcaagac

SEQ ID NO: 33 - DNA, 27 bp, Artificial Sequence
  Primer
cccgctcgagctgcttggcggcaagac

SEQ ID NO: 34 - DNA, 27 bp, Artificial Sequence
  Primer
cccgctcgagctgcttggcggcaagac

SEQ ID NO: 35 - DNA, 31 bp, Artificial Sequence
  Primer
cgcggatcccatatggtcgccgccgacatcg

SEQ ID NO: 36 - DNA, 27 bp, Artificial Sequence
  Primer
cccgctcgagttgcttggcggcaagac

SEQ ID NO: 37 - DNA, 31 bp, Artificial Sequence
  Primer
cgcggatcccatatggtcgccgccgacattg

SEQ ID NO: 38 - DNA, 27 bp, Artificial Sequence
  Primer
cccgctcgagttgcttggcggcaagac

SEQ ID NO: 39 - DNA, 31 bp, Artificial Sequence; identical to SEQ ID NO: 35
  Primer

SEQ ID NO: 40 - DNA, 27 bp, Artificial Sequence
  Primer
cccgctcgagctgcttggcggcaagac

SEQ ID NO: 41 - DNA, 65 bp, Artificial Sequence
  Primer
cgcggatcccatatgggccctgattctgaccgcctgcagcagcggagggtcgccgccgacatcgg

SEQ ID NO: 42 - DNA, 28 bp, Artificial Sequence
  Primer
cccgctcgagctgtttgccggcgatgcc

SEQ ID NO: 43 - DNA, 28 bp, Artificial Sequence
  Primer
gcccaagcttctgtttgccggcgatgct